(19) United States
(12) Patent Application Publication
(10) Pub. No.: US 2023/0414106 A1
(43) Pub. Date: Dec. 28, 2023

(54) System, Method, and Apparatus for Detecting Life/Passing of a Being

(71) Applicant: Detection Innovation Group, Inc., New Port Richey, FL (US)

(72) Inventors: David Segal, Palm Harbor, FL (US); Chris Defant, New Port Richey, FL (US); Steve Chapin, PALM HARBOR, FL (US); Jeremy Nelson, Spring Hill, FL (US); Christopher Jeffers, Trinity, FL (US)

(73) Assignee: Detection Innovation Group, Inc., New Port Richey, FL (US)

(21) Appl. No.: 17/849,750

(22) Filed: Jun. 27, 2022

(51) Int. Cl. A61B 5/01; G01P 13/00; G01P 15/18; G01J 5/00; A61B 5/11; A61B 5/00

(52) U.S. Cl. CPC A61B 5/01; G01P 13/00; G01P 15/18; G01J 5/0025; A61B 5/11; A61B 5/0008; A61B 5/681; A61B 2562/0219

(57) ABSTRACT

A method and apparatus of detecting life in a wearer of a body-worn device includes at least a temperature sensor and a motion sensing device (e.g., an accelerometer) interfaced or within the body-worn device, each coupled to the wearer of the body worn device to measure body temperature of the wearer as well as movement of the wearer. Further sensors such as heart beat sensors are also anticipated for improved accuracy. The method and apparatus of detecting life declares that the wearer is dead after successive periodic readings of temperature have continually changed at a predetermined rate and no movement has been detected by the motion sensing device for a predetermined period of time. In some embodiments, the motion sensing device is a three-axis accelerometer.

### FIELD

This invention relates to the field of health and more particularly to a system for detecting when a being that is wearing a device has died.

### BACKGROUND

There are many situations in which it is desired or required to determine if a wearer (e.g., a being, human or animal) of a device is alive or has passed. For example, in various senior living establishments, it is prudent to dispatch help when it is detected that a wearer of the device has passed. On the other hand, there is a need to reduce or eliminate false alarms due to a malfunctioning device, battery failure, interferences (e.g., radio interference or sensor interference), or removal by the wearer. For example, in the senior living establishment, if the device is a smart watch that only detects heartrate and alarms if the heartrate drops below a certain threshold, then an alarm will be triggered when the wearer takes off the smart watch when bathing.

Another use for monitoring life or passing of a being is within prison complexes. In such facilities, it is important to monitor and count all inmates to provide a reliable headcount to assure that no inmate is missing. Currently, headcounts are performed manually, often every few hours, often requiring inmates to gather in one location for counting. In many prisons, it is required that there be between six and ten headcounts taken per day. For each headcount, it must be ascertained whether the person being counted is actually alive.

Recently, in such facilities, inmates are often outfitted with locking and tamper-resistant body-worn devices, usually attached to an appendage, for example, at a wrist or ankle. Again, it is desired that a false alarm not be initiated as might occur when the inmate is in an area where RF signals cannot penetrate, for example, when an inmate is inside a walk-in freezer.

Unfortunately, it is difficult to reliably determine if the person is alive only from temperature. For example, in a system that uses only temperature compared to a fixed threshold, if the person's temperature drops below 97 degrees Fahrenheit an alarm is triggered. The inmate who is working in the walk-in freezer may experience a body temperature at an appendage of less than 97 degrees upon exiting, depending upon how long they are in the walk-in freezer, clothing, and the temperature in the walk-in freezer. A similar situation occurs when inmates are out in the yard during very cold temperatures. Further, being that these body-worn devices are designed to be worn by the inmate at all times, parameters such as temperature will vary during bathing.

What is needed is a system that will reliably detect when a wearer of the system is alive.

### SUMMARY

The system for detecting life of a being is worn by the being (e.g., a person) and is either free for removal or locked to the being. The system for detecting life of a being uses multiple sensors that, correlated together, provide an accurate status of life that can be relied upon in many scenarios, including senior living facilities and prison facilities. The system for detecting life of a being utilizes at least two measurements in order to accurately distinguish a live wearer from a dead wearer. In some embodiments, one of the measurements is temperature, but not absolute temperature as absolute temperature is dependent upon the being (e.g., some people have lower normal temperatures) and absolute temperature is affected by ambient temperature and clothing. Instead, the system for detecting life of a being utilizes change in temperature over time as one indication of death of the being. It is known that after death, a being will gradually change at a rate dependent of the ambient temperature around the body (note that in a desert in 110 degree ambient, a dead body's temperature will rise), the weight/mass of the body, body-fat content, and insulation around the body such as clothing, but in no case will the temperature change be sudden as when the body-worn device is removed or immersed in water. Forensically, the body changing rate is often used to determine an exact time of death, taken in relation to the above parameters. Depending upon ambient temperature and the parameters above, in cooler temperatures, the body temperature at the skin might drop steadily, for example, by from 0.75 degrees Fahrenheit to 1.5 degrees Fahrenheit per time period for several hours after death. By periodically measuring body temperature at the skin, it can be determined that the body temperature at the skin has decreased steadily over sufficient time to declare death.

To further add accuracy to this determination, it has been observed that beings cannot remain motionless for more than a certain period of time, even when sleeping. For humans, it has been determined that one cannot go without moving longer than sixteen minutes, but a dead body is typically motionless unless acted upon by outside forces. Therefore, using a motion sensor such as an accelerometer, a second determination of death is made when it is detected that the wearer has not moved for greater than a predetermined time period, for example, sixteen or twenty minutes. For such, any electronic accelerometer is anticipated, but preferably a three-axis accelerometer.

By combining the measurement of temperature over time with detecting movement over time, if the being has a temperature that is consistently decreasing over a period of time and no movement has been detected from that being for the predetermined time period, it can be reliably determined that the being is no longer alive. Additional sensors such as tamper detectors, heart rate detectors (pulse), etc., are further anticipated to further increase the reliability of the determination of death while further reducing the probability of a false alarm, even when removal of the device occurs, the device is submerged in water, the wearer experiences ambient temperature extremes, etc. Such determination of death/life is sufficiently accurate that this determination is usable in automated head counting of prison inmates.

In one embodiment, a system for detecting life of a being is disclosed including a base station that has a base station processor and a base station transceiver that is operatively coupled to the base station processor. A body-worn device includes a processor, a temperature sensor operatively coupled to the processor, an accelerometer operatively coupled to the processor, a transceiver operatively coupled to the processor, and a source of power. The source of power provides operational power to the processor, and to the transceiver while the temperature sensor is configured to measure a body temperature of a wearer of the body-worn device. The processor of the body-worn device records a time of last movement each time a signal of movement is received from the accelerometer. The processor periodically reads the body temperature of the wearer from the temperature sensor and records the body temperature in a histogram. When analysis of the body temperatures recorded in the histogram indicate a decrease in body temperature of greater than a predetermined rate for a predetermined time period and the last time of movement is greater than a predetermined movement time from a current time, the processor determines that the wearer of the body-worn device is dead.

In another embodiment, a system for detecting life of a being is disclosed. The system includes a base station that has a base station processor and a base station transceiver that is operatively coupled to the base station processor. A body-worn device includes a processor, a temperature sensor operatively coupled to the processor, an accelerometer operatively coupled to the processor, a transceiver operatively coupled to the processor, and a source of power. The source of power provides operational power to the processor and to the transceiver. The temperature sensor is configured to measure a body temperature of a wearer of the body-worn device (e.g., an infrared temperature sensor aimed at the wearer or a contact temperature sensor in contact with the wearer). The processor of the body-worn device records a time of last movement each time a signal of movement is received from the accelerometer and periodically reads the body temperature of the wearer from the temperature sensor and transmits the body temperature of the wearer and the time of last movement from the transceiver to the base station transceiver. Upon the base station processor receiving the body temperature and the time of last movement, the base station processor records the body temperature (e.g., in a histogram) and the base station processor analyzes the histogram to determine if the wearer of the body-worn device is dead when there was a decrease in body temperature of greater than a predetermined rate for a predetermined time period and the last time of movement is greater than a predetermined movement time from a current time.

In another embodiment, a method of determining if a wearer of a body worn device is dead is disclosed. The body worn device has a temperature sensor operatively coupled to the wearer and measuring a body temperature of the wearer and the body-worn device has an accelerometer. The method includes (a) using the accelerometer to continuously record a time of last movement of the body-worn device and to (b) periodically read the body temperature from the temperature sensor. Analysis is performed to (c) determine a status of the wearer of the body-worn device as being dead when the body temperature of the wearer has decreased by at least a predetermined amount over a predetermined period of time and the time of last movement is greater than a predetermined time from the current time.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a schematic view of multiple body-worn devices in a network of the system for detecting life of a being.

FIG. 2 illustrates a block diagram of an exemplary tamper-resistant body-worn device.

FIG. 3 illustrates a block diagram of an exemplary removable body-worn device.

FIG. 4 illustrates a perspective view of the exemplary tamper-resistant body-worn device.

FIG. 5 illustrates a plan view of the exemplary removable body-worn device.

FIG. 6 illustrates a data connection diagram of the system for detecting passing of a being.

FIG. 7 illustrates an exemplary data record of a body-worn device.

FIG. 8 illustrates a second exemplary data record of the body-worn device.

FIG. 9 illustrates a flow chart of exemplary software of a body worn-device of the system for detecting life of a being.

FIG. 10 illustrates a flow chart of exemplary software of a base station of the system for detecting life of a being.

FIG. 11 illustrates an alternate flow chart of exemplary software of a body worn-device of the system for detecting life of a being.

FIG. 12 illustrates an alternate flow chart of exemplary software of a base station of the system for detecting life of a being.

FIG. 13 illustrates a flow chart of exemplary software of a base station performing a headcount of the system for detecting life of a being.

FIG. 14 illustrates a schematic view of an exemplary body worn-device.

FIG. 15 illustrates a schematic view of an exemplary system of a base station.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

The described system pertains to a collection of hardware devices for monitoring biological data related to any target person and for reliably determining the target person's status being either alive or dead. For brevity and clarity reasons, throughout this description, the target person is typically a detained person such as an inmate in a correctional facility or person on house arrest, but there is no restriction to any particular type of target person, nor that the target be a human being, in that, the described body-worn device functions the same for any animal or human. The described system is equally applicable to any other type of scenario. For example, the target person is a person living in an assisted care facility and the body worn device is worn by the person to monitor that person's current status.

For simplicity purposes, the following description uses, as an example, an inmate as the target person. In general, depending upon security and policies at a prison, the population (inmates) must be counted (headcount) periodically throughout each day. According to such procedures, in the past, inmates were required to move to an area to be physically counted by prison personnel (e.g., guards).

Throughout this description, a body-worn device is shown as a tamper-resistant body-worn device (e.g., the type of body-worn device that is locked onto an inmate or any person such as a person on house arrest or parole) or a removable body-worn device (e.g., a body-worn device that is in contact with the user, typically a smartwatch).

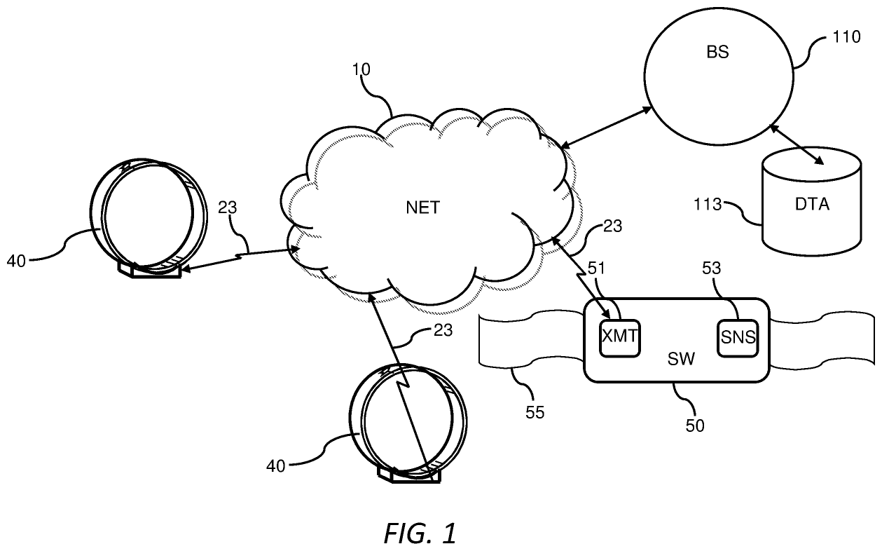

Referring to FIG. 1, a schematic view of multiple body-worn devices in a network of the system for detecting life of a being is shown. In this, several tamper-resistant body-worn devices 40 and a removable body-worn device 50 are shown connected to a base station 110 through a wireless network 10, though systems with only tamper-resistant body-worn devices 40 or only removable body-worn device 50 are equally anticipated. The overall structure, communication paths, and connection relationships shown are one example of a wireless communication system and are not meant to limit this disclosure in any way. Many different organizations, protocols, operating frequencies (bands), and architectures are anticipated and all of which are included here within. The tamper-resistant body-worn devices 40 and removable body-worn devices 50 are intended to operate with any known network 10, including the cellular network and all known and future wireless networks or point-to-point wireless systems. Wireless networks, are for example, the cellular phone network (e.g., GSM, 4G, 5G, CDMA, AMPS), wireless local area networks (e.g., WiFi-802.11x), etc. Wireless networks also include point-to-point systems such as Bluetooth, and any other licensed or unlicensed forms of wireless communications. In the system shown in FIG. 1, the tamper-resistant body-worn devices 40 and removable body-worn devices 50 periodically communicate with the base station 110 through the network 10 to convey various data that is obtained from the tamper-resistant body-worn devices 40 and removable body-worn devices 50 such as detection of tampering and biological data regarding the wearer of such.

Throughout this description, the tamper-resistant body-worn devices 40 and removable body-worn devices 50 will be collectively referred to as body-worn devices 40/50, representing any combination and type of a collection of such devices.

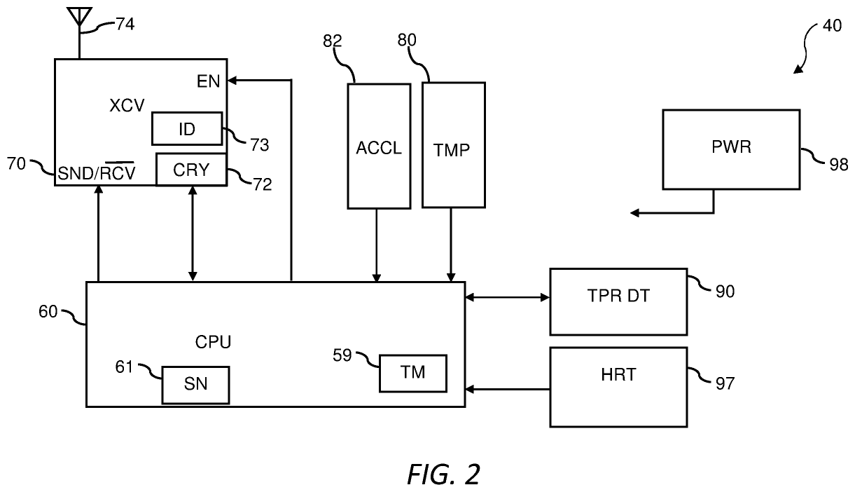

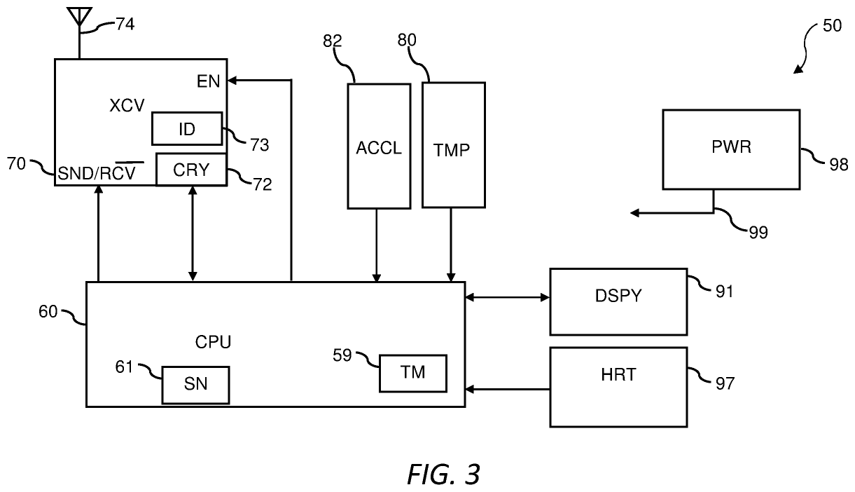

Within the body-worn devices 40/50 is circuitry (see FIGS. 2 and 3) that implements the various features of the body-worn devices 40/50, including some or all of communications with the base station 110, tamper detection, measuring biological data, and providing power for the above.

Referring to FIG. 2, a block diagram of the tamper-resistant body-worn device 40 is shown. Although the exemplary block diagram of the tamper-resistant body-worn device 40 is shown being processor-based, it is equally anticipated that the same or similar functionality be implemented in other forms of electronics such as discrete logic or logic arrays.

The circuitry of the tamper-resistant body-worn device 40 includes a source of power 98. It is well known how to power such devices ranging from miniature body worn devices such as watches to more complicated devices that are often specialized worn devices such as house-arrest tracking devices. Any source(s) of power are anticipated, including, but not limited to, batteries, rechargeable batteries, solar cells, radio frequency parasitic extraction, capacitors, super capacitors, fuel cells, etc., including combinations of such. The source of power 98 includes circuitry to condition and regulate the power which is then distributed to the various subsystems 60/70/80/90 as known in the industry. In some embodiments, the source of power 98 further includes circuitry to control charging as well as a connection or interface to a source of charging power (e.g., a wall-wart, base station, etc.).

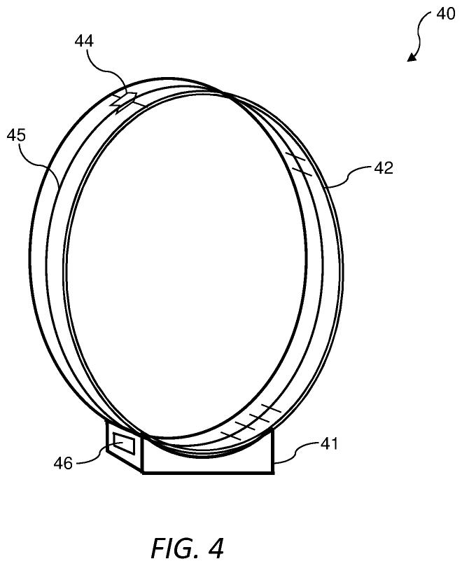

A tamper detection subsystem 90 is interfaced to the processor 60. The processor 60 controls the operation of the tamper detection subsystem 90 by sending commands and/or signals to the tamper detection subsystem 90 and receiving status and data back (e.g., “intact” or “device removed from body,” etc.). It is anticipated that the tamper-resistant body-worn device 40 is issued to a particular individual (e.g., inmate) and is locked onto that person by, for example, a leg cuff, arm cuff, neck cuff, belt, etc. Although the tamper-resistant body-worn device 40 is secured to the person and not easily removed, it is important that any tampering of the tamper-resistant body-worn device 40 be detected (and reported). There are many methods of detecting tampering or removal of a body worn device 40 known in the industry, all of which are anticipated and included here within. For example, in some embodiments, a conduction path fully encircles the body appendage to which the body worn device 40 is attached such that, if the enclosure 41 (see FIG. 4) is cut, the circuit opens and the open circuit is detected by the tamper detection subsystem 90. This is a somewhat simple method that is used as an example; in that, a clever person can expose the conductor in two locations, attach an end of a wire to the conductor in each location, then cut through the strap in between the two locations without detection. In some embodiments, more elaborate measurements are used to detect the added resistance (or change in resistance) of the external wire. In some embodiments, an optical light pipe connected at both ends to the tamper-resistant body-worn device 40 encircles the appendage and a particular wavelength(s) of light or an encoded light wave signal is emitted into one end of the light pipe. If the signal is detected at the other end, then it is believed that no tampering has occurred, but if the signal is not detected, then tampering is detected and an appropriate alert is transmitted as will be described. There are many types of tamper detection devices anticipated including the above and/or any other type of tamper detection including, but not limited to, motion sensors and accelerometers 82 (e.g., if no movement is detected for a long period of time, it is assumed that the tamper-resistant body-worn device 40 has been removed from the body). Note that in some embodiments, the accelerometers 82 are three-axis accelerometers.

In some embodiments, the tamper detection subsystem 90 also includes intrusion detection to determine if the enclosure 41 (see FIG. 4) around the electronics has been penetrated. Again, there are many ways to detect such intrusion as known in the industry, all of which are included here within. For example, a simple method includes a micro switch that detects opening of a cover of the enclosure 41, or the detection of light within the enclosure 41 (see FIG. 4). Normally, there is no light within the enclosure 41 being that the enclosure 41 is made of a non-light transmitting material and completely sealed with no openings, but when the enclosure 41 is compromised, light is allowed to enter the enclosure 41 and triggers the tamper detection subsystem 90. In other embodiments, there is an internal detector that detects one or more materials or physical state normally present in the atmosphere (e.g., change in pressure, humidity, oxygen, nitrogen, etc.) and the enclosure 41 is either evacuated or filled with some other gas (e.g., helium). In this, normally, the detector measures presence or absence of the material, but when the enclosure 41 is cut, atmosphere enters the housing, the gain or loss of the material is detected, and the tamper detection subsystem 90 is triggered.

In some embodiments, the tamper-resistant body-worn device 40 communicates with the base station(s) 110 through a wireless transceiver preferably having an antenna 74. The wireless transceiver 70 is interfaced to the processor 60 and the processor 60 communicates with and controls the operation of the wireless transceiver 70 by sending commands and data to the wireless transceiver 70 and receiving status and data back in a similar manner. Because such wireless transceivers 70 often consume significant power, in some embodiments, the processor 60 has an enable interface to power down the wireless transceiver 70 (or any other subsystem) when not in use. Any appropriate signaling protocol is anticipated, as transmission collisions with other body-worn devices lost packets, out-of-order packets, noise, etc., must be overcome. The data and signaling is modulated onto a radio frequency using any modulation format such as frequency modulation, amplitude modulation, pulse code modulation, pulse width modulation, etc.

It is anticipated that the wireless transceiver 70 be any type of transceiver, operating over any known frequency or group of frequencies, any known power level(s), and either half-duplex or full-duplex. When the wireless transceiver 70 is half-duplex, the processor 60 controls whether the wireless transceiver 70 is receiving or it is transmitting by a mode control.

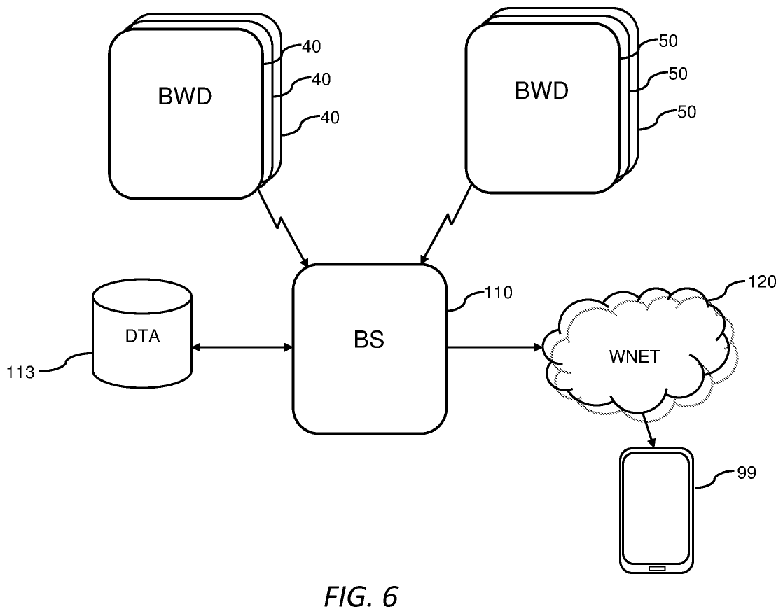

Data is transferred between the processor 60 and the wireless transceiver 70 in any way known in the industry including, but not limited to, shared memory (not shown), serial transfer, parallel transfer, any combination, etc. In some embodiments, though not required, data from the processor 60 is encrypted before transmission. In such, the data is either encrypted by instructions running on the processor 60, or, in some embodiments, by an encryption module 72 within or external to the wireless transceiver 70. Also, in some embodiments, though not required, data from the base station 110 (see FIG. 6) is encrypted before transmission. In such, the encrypted data is received by the wireless transceiver 70, and then the encrypted data is either decrypted by instructions running on the processor 60, or, in some embodiments, by the hardware encryption module 72 within or external to the wireless transceiver 70.

Any band, frequency, wavelength, set of wavelengths, protocols, protocol stacks are anticipated for use by the wireless transceiver 70 to communicate with the base station(s) 110. There are many protocols and protocol options that provide various transmission capabilities to improve reliability of communications, reduction or elimination of transmission errors, and/or efficiencies in both spectrum usage as well as power consumption.

In some embodiments, a clock or timekeeper 59 is included, either as a subsystem of the processor 60 or a separate, discrete timing device that is interface to the processor 60. In such embodiments, the tamper-resistant body-worn device 40 has the ability to record the time and/or date of any event and to transmit the time and/or date to the base station 110 along with any alert and/or data.

In some embodiments, the wireless transceiver 70 includes an identification 73 that uniquely distinguishes the wireless transceiver 70 from other wireless transceivers 70 so that when the base station 110 receives a packet of data from the wireless transceiver 70, the base station 110 is able to correlate that packet of data to the wearer of the body-worn device 40/50. In some embodiments, the processor 60 has access to a serial number 61 and encodes the serial number 61 into each packet transmitted for the correlation of each packet of data to the wearer of the body-worn device 40/50.

When tampering is detected by the tamper detection subsystem 90, the processor 60 receives an indication of such and transmits an alert to the base station 110 through the network 10.

Other data gathered from sensors such as an acceleration sensor 82, a temperature sensor 80, and a heart beat sensor 97 are obtained by the processor 60 and sent to the base station 110 through the network 10 either periodically or when polled by the base station 110. As will be shown, this data is used to determine a status of the wearer of the tamper-resistant body-worn device 40 (e.g., alive or dead). Note that, in some embodiments, the determination of status (live or dead) is performed by the processor 60 and conveyed to the base station 110 either periodically, when a change to the status occurs, or when polled by the base station 110. In other embodiments, the data is periodically read and transmitted to the base station 110 and the base station 110 calculates the status (live or dead).

Referring to FIG. 4, a block diagram of the removable body-worn device 50 is shown. Although the exemplary block diagram of the removable body-worn device 50 is shown being processor-based, it is equally anticipated that the same or similar functionality be implemented in other forms of electronics such as discrete logic or logic arrays.

The circuitry of the removable body-worn device 50 includes a source of power 98. It is well known how to power such devices ranging from miniature body worn devices such as watches to more complicated devices that are often specialized worn devices such as house-arrest tracking devices. Any source(s) of power are anticipated, including, but not limited to, batteries, rechargeable batteries, solar cells, radio frequency parasitic extraction, capacitors, super capacitors, fuel cells, etc., including combinations of such. The source of power 98 includes circuitry to condition and regulate the power which is then distributed to the various subsystems 60/70/80/90 as known in the industry. In some embodiments, the source of power 98 further includes circuitry to control charging as well as a connection or interface to a source of charging power (e.g., a wall-wart, base station, etc.).

In some embodiments, the removable body-worn device 50 communicates with the base station(s) 110 through a wireless transceiver 70, preferably having an antenna 74. The wireless transceiver 70 is interfaced to the processor 60 and the processor 60 communicates with and controls the operation of the wireless transceiver 70 by sending commands and data to the wireless transceiver 70 and receiving status and data back in a similar manner. Because such wireless transceivers 70 often consume significant power, in some embodiments, the processor 60 has an enable interface to power down the wireless transceiver 70 (or any other subsystem) when not in use. Any appropriate signaling protocol is anticipated, as transmission collisions with other body-worn devices 40/50, lost packets, out-of-order packets, noise, etc., must be overcome. The data and signaling is modulated onto a radio frequency using any modulation format such as frequency modulation, amplitude modulation, pulse code modulation, pulse width modulation, etc.

It is anticipated that the wireless transceiver 70 be any type of transceiver, operating over any known frequency or group of frequencies, any known power level(s), and either half-duplex or full-duplex. When the wireless transceiver 70 is half-duplex, the processor 60 controls whether the wireless transceiver 70 is receiving or it is transmitting by a mode control.

Data is transferred between the processor 60 and the wireless transceiver 70 in any way known in the industry including, but not limited to, shared memory (not shown), serial transfer, parallel transfer, any combination, etc. In some embodiments, though not required, data from the processor 60 is encrypted before transmission. In such, the data is either encrypted by instructions running on the processor 60, or, in some embodiments, by an encryption module 72 within or external to the wireless transceiver 70. Also, in some embodiments, though not required, data from the base station 110 (see FIG. 6) is encrypted before transmission. In such, the encrypted data is received by the wireless transceiver 70, and then the encrypted data is either decrypted by instructions running on the processor 60, or, in some embodiments, by the hardware encryption module 72 within or external to the wireless transceiver 70.

Any band, frequency, wavelength, set of wavelengths, protocols, protocol stacks are anticipated for use by the wireless transceiver 70 to communicate with the base station(s) 110. There are many protocols and protocol options that provide various transmission capabilities to improve reliability of communications, reduction or elimination of transmission errors, and/or efficiencies in both spectrum usage as well as power consumption.

In some embodiments, the wireless transceiver 70 includes an identification 73 that uniquely distinguishes the wireless transceiver 70 from other wireless transceivers 70 so that when the base station 110 receives a packet of data from the wireless transceiver 70, the base station 110 is able to correlate that packet of data to the wearer of the body-worn device 40/50 (e.g., a MAC address as known in the industry). In some embodiments, the processor 60 has access to a serial number 61 and encodes the serial number 61 into each packet transmitted for the correlation of each packet of data to the wearer of the body-worn device 40/50.

In some embodiments, a clock or timekeeper 59 is included, either as a subsystem of the processor 60 or a separate, discrete timing device that is interface to the processor 60. In such embodiments, the removable body-worn device 50 has the ability to record the time and/or date of any event and to transmit the time and/or date to the base station 110 along with any alert and/or data.

Other data gathered from sensors such as an acceleration sensor 82, a temperature sensor 80, and a heartbeat sensor 97 are obtained by the processor 60 and sent to the base station 110 through the network 10 either periodically or when polled by the base station 110. As will be shown, this data is used to determine a status of the wearer of the removable body-worn device 50 (e.g., alive or dead).

Note that, in some embodiments, the determination of status (live or dead) is performed by the processor 60 and conveyed to the base station 110 either periodically, when a change to the status occurs, or when polled by the base station 110. In other embodiments, the data is periodically read and transmitted to the base station 110 and the base station 110 calculates the status (live or dead).

The removable body-worn device 50 differs from the tamper-resistant body-worn device 40, in that the removable body-worn device 50 typically has a display 91 (e.g., a clock face or any digital display) and the tamper-resistant body-worn device 40 has the tamper-detection subsystem 90 that is not present in the removable body-worn device 50 as it is anticipated that the removable body-worn device 50 be removed from the user's appendage periodically, for example, during bathing and sleep.

Referring to FIG. 4, a perspective view of an exemplary tamper-resistant body-worn device 40 is shown. In this example, the tamper-resistant body-worn device 40 is a collar, such as a leg collar, arm collar, or neck collar, while in other embodiments; the body worn device 40 is of slightly different forms for attachment to the body in different ways such as by a belt-like system. In this exemplary tamper-resistant body-worn device 40, some or all of the circuitry is located within an enclosure 41 that is made as part of the strap 42 or affixed to the strap 42 so as to resist removal and/or intrusion. The strap 42 is locked closed after placing around the person's appendage, for example by a tamper-proof lock 44. In some embodiments, the lock 44 is part of the enclosure 41. In some embodiments, the tamper-proof lock includes a one-way closure system in which, the strap 42 is tightened around an appendage by capturing more of the strap 42 through the one-way closure system, then cutting off any excess of the strap 42. In some embodiments, especially those with electronics, conductors, and/or light pipes within the strap 42, the strap 42 is of fixed length and locks into the enclosure 41, completing the tamper detection circuit. In the industry of inmate or release monitoring (e.g., within prisons or for house arrest), it is well known how to attach a tamper-resistant body-worn device 40 to a person and to detect tampering and/or removal, all of which are anticipated and included here within.

Although any form of attachment mechanism is anticipated for the tamper-resistant body-worn device 40, in some embodiments, the attachment mechanisms and enclosure 41 are designed to prevent removal under normal wear and impact that often occurs during the wearing of such device such as, during exercise, walking, running, etc. Furthermore, in some embodiments, the attachment mechanisms and enclosure 41 are designed to resist penetration by substances that normally contact the wearer such as during showering, rain, etc. Although any suitable material is anticipated, it is preferred that at least the surface of the strap 42 and/or enclosure 41 be made from a hypoallergenic material such as Santoprene, being that the body worn device 40 will be worn for long periods of time. It is also preferred that the strap 42 be made from materials that will not significantly stretch, even when heated. Stretching is not desired because, in some cases, stretching enables easy removal without detection of tampering. In some embodiments, the enclosure 41 is made of an impact resistant polycarbonate that is rugged, tamper resistant, and seals the electronics from the surrounding environment.

As previously described, in some embodiments, the tamper-resistant body-worn device 40 includes a perimeter detection loop 45 that consists of a conductor (either light or electrical signal) that helps detect tampering. For example, if the strap 42 is cut, the perimeter detection loop 45 is broken and a tamper signal is sent from the wireless transceiver 70 of the tamper-resistant body-worn device 40 to the base station 110.

In some embodiments, an RFID 46 is mounted in/on the enclosure 41 and/or in the strap 42. This optional RFID (or other readable mechanism such as a bar code, QR code, etc.) is available for use to interment facility for many uses such as head counts, usage accounting, commissary expense charges, etc.

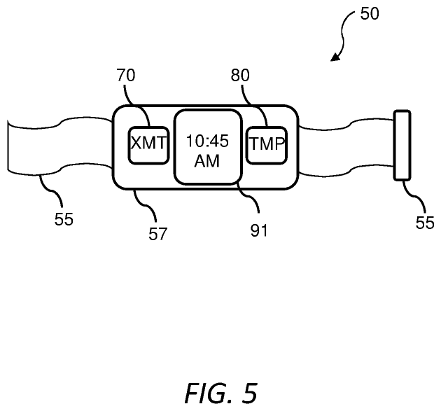

Referring to FIG. 5, a perspective view of an exemplary removable body-worn device 50 is shown. In this example, the removable body-worn device 50 is a smartwatch that attaches to the body by a strap and clasp system 55. In this exemplary removable body-worn device 50, some or all of the circuitry (e.g., processor 60, transceiver 70, temperature sensor 80, accelerometer 82) is located within a watch enclosure 57 that is made as part of the strap and clasp system 55 or affixed to the strap and clasp system 55. The strap and clasp system 55 hold the removable body-worn device 50 onto the person after placing around the person's appendage.

The removable body-worn device 50 is shown including the transceiver 70 for communicating with the base station 110 and sensors (e.g., the accelerometer 82, temperature sensor 80, and heartbeat sensor 97) for reading biometric parameters from the wearer of the removable body-worn device 50. For completeness, the removable body-worn device has a display 91 for communicating with the wearer information such as received text messages, missed call information, and the time-of-day, as does many smart watches.

Referring to FIG. 6, a block diagram of communications between a body-worn device 40/50 and a base station 110 is shown.

In some embodiments, the condition of the battery in the body worn device 40 is also reported during some or all transmissions. In some embodiments, diagnostics or self-tests are performed during initialization and/or periodically and any anomalies are reported through the wireless interface between the wireless transceiver 70 and the base station 110.

As will be shown, the base station 110 has access to data 113 regarding the population of users, relating the id 73 and/or serial number 61 to each individual in the population so that when data is received from a particular body-worn device 40/50, the data is correlated to an associated user in the population of users and logged as needed.

In one scenario, when data is received from a particular body-worn device 40/50 indicative of the user passing, an alert is issued. Such an alert is anticipated to by any electronic/visual/audible alert as known in the industry. In addition, in some embodiments, an alert is sent through a wide-area network 120 (e.g., phone system, cellular system) to a remote device 99 (e.g., a smartphone or telephone) to indicate the passing so that the user can be located and helped or taken care of in whatever way procedures indicate.

The following examples use a fictitious inmate, John Doe, as an example of a person assigned and wearing a body-worn device 40/50. Note that the disclosed inventions are in any way limited to prisons or correctional facilities.

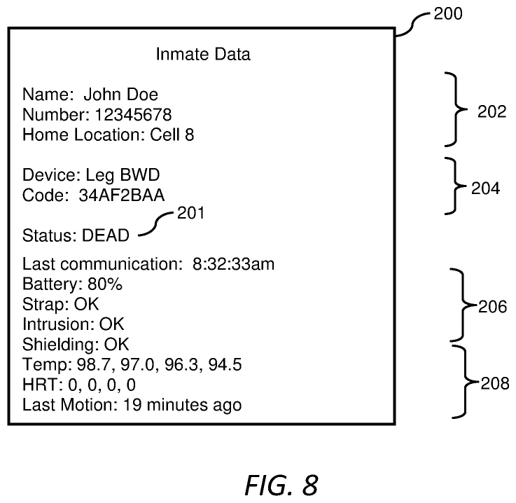

Referring to FIG. 8, an exemplary user interface 200 showing the status of a body-worn device 40/50 is shown. In this example, data pertaining to the person 202 includes an inmate name (John Doe), an inmate number (12345678), and a home location (Cell 8). Identification data 204 pertaining to the body-worn device 40/50 assigned to this inmate includes a description of the device (Leg BWD) and a code (34AF2BAA) which is, for example, a serial number 61 or identification 73 of this body-worn device 40/50.

Next, status 201 is shown/displayed (in this example, alive) along with data 206 regarding the assigned body-worn device 40/50, including a time of last communication, a condition of the battery, whether the body-worn device 40/50 has an indication of a broken strap, intrusion into the electronics, or has been cloaked. Note that, in some embodiments, more or less information is included/displayed.

In some embodiments, historical data 208 is shown/displayed as logged from the body-worn device 40/50 such as the prior temperature readings, the prior heart rate readings, and the time since last motion was detected by the accelerometer 82. Note that in some embodiments, more or less sensors 80/82/97 or other sensors are employed to detect life and the present disclosure is in no way limited to any particular combination of biometric data. As can be inferred from the prior temperature readings (98.7, 98.5, 98.7, 98.8), the prior heart rate readings (63, 63, 60, 58), and the time since last motion was detected (3.8 minutes ago), the wearer of this body-worn device 40/50 is determined to be alive.

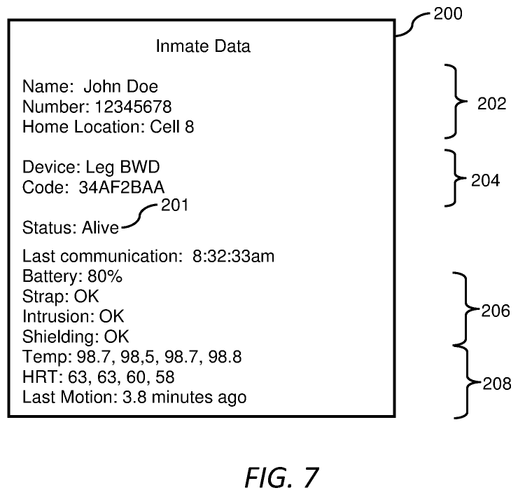

Referring to FIG. 8, the exemplary user interface 200 of FIG. 7 at a later time showing a different status of a body-worn device 40/50. As above, data pertaining to the person 202 is displayed includes the inmate's name (John Doe), the inmate number (12345678), and the home location (Cell 8). As above, identification data 204 pertaining to the body-worn device 40/50 assigned to this inmate includes a description of the device (Leg BWD) and a code (34AF2BAA) which is, for example, a serial number 61 or identification 73 of this body-worn device 40/50.

Next, status 201 is shown/displayed (in this example, DEAD) along with data 206 regarding the assigned body-worn device 40/50, including a time of last communication, a condition of the battery, whether the body-worn device 40/50 has an indication of a broken strap, intrusion into the electronics, or has been cloaked. Note that, in some embodiments, more or less information is included/displayed.

In some embodiments, historical data 208 is shown/displayed as logged from the body-worn device 40/50 relating to the status such as the prior temperature readings, the prior heart rate readings, and the time since last motion was detected by the accelerometer 82. Note that in some embodiments, more or less sensors 80/82/97 or other sensors are employed to detect life and the present disclosure is in no way limited to any particular combination of biometric data. As can be inferred from the prior temperature readings per the present invention (98.7, 97.0, 96.3, 94.5) steadily decreasing, the prior heart rate readings (0, 0, 0, 0), basically no heart rate, and the time since last motion was detected (19 minutes ago), the wearer of this body-worn device 40/50 is determined to be DEAD by the algorithms of the present invention.

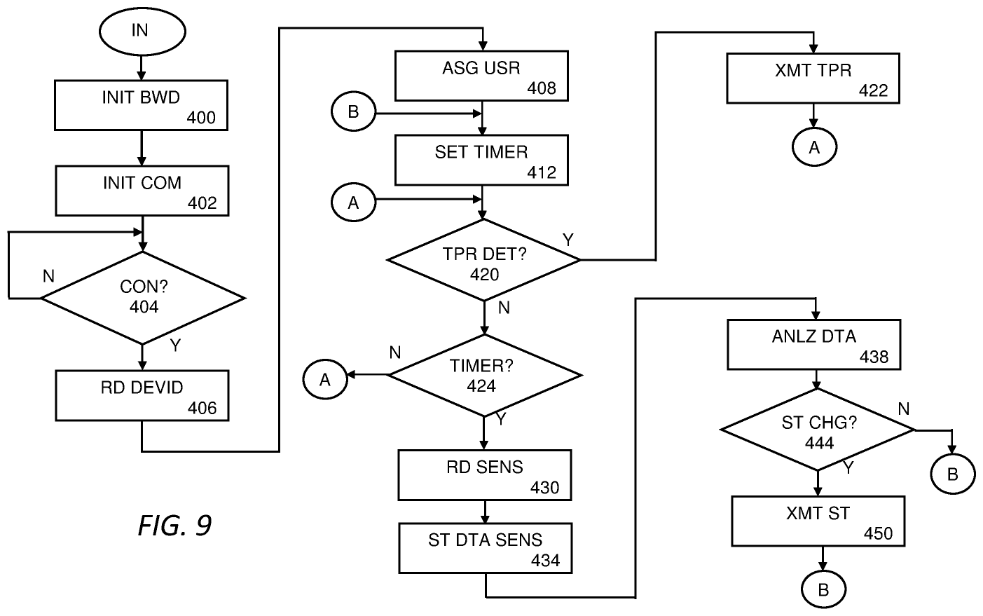

Referring to FIG. 9, a flow chart of exemplary software of a body worn-device 40/50 of the system for detecting life of a being is shown. When power is initially applied to the body worn-device 40/50, the processor 60 initializes 400 and then initializes communications 402 to the base station 110. The system repeatedly attempts to communicate with the base station 110 until a connection is detected 404, at which time the identification 73 or serial number 61 of the body worn-device 40/50 is read 406 and transmitted to the base station 110 to correlate (or assign 408) the identification 73 or serial number 61 to the wearer that is assigned the body worn-device 40/50, although any known mechanism for correlating the body worn-device 40/50 to the wearer is fully anticipated. The base station 110 stores the identification 73 or serial number 61 of the body worn-device 40/50, such that, any future communications containing that identification 73 or serial number 61 of the body worn-device 40/50 will correlate to that user (e.g., inmate).

Now, the body worn-device 40/50 continuously loops (B), each time through the loop a timer is set 412 and a sub-loop (A) starts with accessing the tamper detection subsystem 90 to determine if tampering has been detected 420. If tampering has been detected 420, a signal or packet indicating that this particular body worn-device 40/50 has been tampered (e.g., removed, broke) 422 is sent to the base station 110.

If tampering has not been detected 420, the timer is checked 424 for expiration and if the timer has not expired, the sub-loop (A) continues. If the timer has expired, the sensors 80/82/97 are read 430 and data from each sensor 80/82/97 is recorded (e.g., stored 434 in memory of the processor 60). The data is analyzed 438 to determine a status of the wearer of the body worn-device 40/50 (e.g., is the wearer of the body worn-device 40/50 alive or dead. If the status hasn't changed 444, the loop (B) continues but if the status has changed 444 (e.g., the wearer of the body worn-device 40/50 was alive and is now dead, the new status is transmitted 450 to the base station 110.

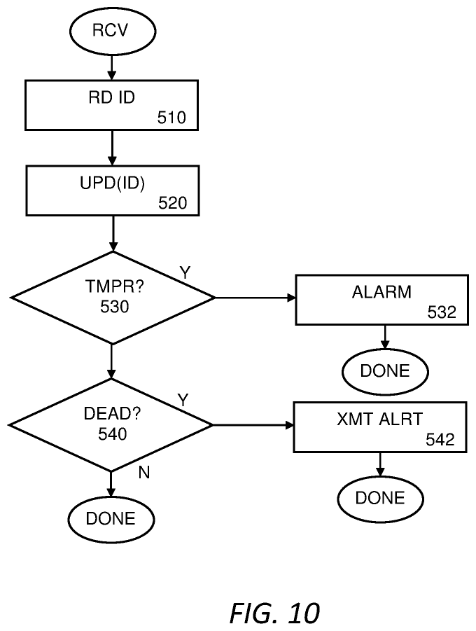

Referring to FIG. 10, a flow chart of exemplary software of a base station 110 of the system for detecting life of a being is shown. In this example, when the base station 110 receives a transmission from any of the population of body worn-devices 40/50, the base station 110 reads 510 the identification of the body worn-devices 40/50 that sent the transmission and updates data 520 regarding that body-worn device 40/50 (e.g., updates sensor data, status, tamper status). The base station then determines if the transmission includes an indication of tampering 530 of the body worn-device 40/50 and if the transmission includes an indication of tampering 530 of the body worn-device 40/50, an alarm is issued 532, for example, an audible alarm, visual alarm, transmission of one or more messages to employees/guards (e.g., SMS messages), etc. If the transmission does not include an indication of tampering 530 of the body worn-device 40/50, then the base station determines if the transmission includes an indication of a change in status 540 of the wearer of the body worn-device 40/50 (e.g., a change from living to dead) and if the transmission includes an indication of the change in status 540, in some embodiments, one or more messages are sent 542 to employees/guards (e.g., SMS messages), etc.

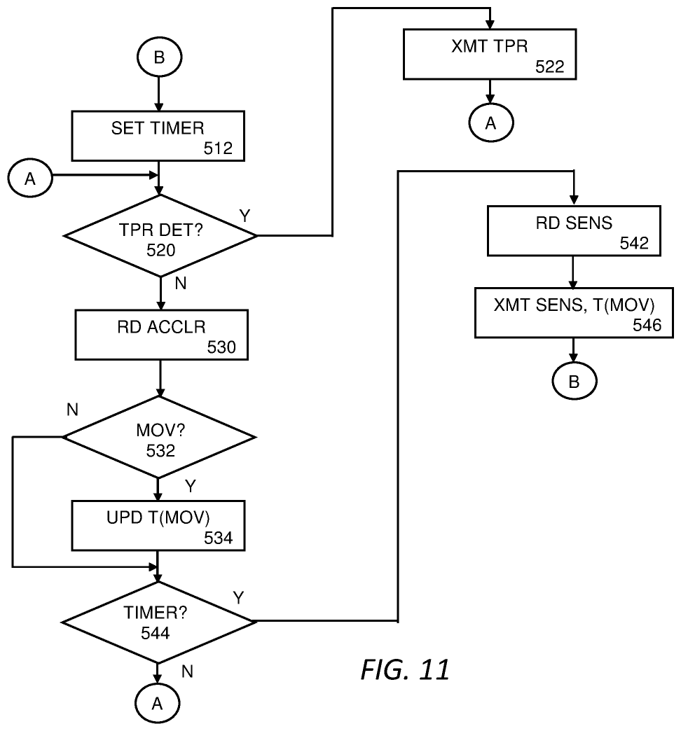

Referring to FIG. 11, an alternate flow chart of exemplary software of a body worn-device 40/50 of the system for detecting life of a being is shown. In this example, the sensor data is periodically read by the processor 60 of the body worn-device 40/50 and transmitted to the base station 110 for analysis. Similar, or any type of initialization steps are performed as in FIG. 9.

Now, the body worn-device 40/50 continuously loops (B), each time through the loop a timer is set 512 and a sub-loop (A) starts with accessing the tamper detection subsystem 90 to determine if tampering has been detected 520. If tampering has been detected 520, a signal or packet indicating that this particular body worn-device 40/50 has been tampered (e.g., removed, broke) is sent 522 to the base station 110.

If tampering has not been detected 520, the accelerometer is read 530 to determine if the wearer of the body worn-device 40/50 has moved and if movement is detected 532, the time of last movement is updated 534. Now, the timer is checked 544 for expiration and if the timer has not expired, the sub-loop (A) continues. If the timer has expired, the sensors are read 542 and data from each sensor 80/82/97 and the time since last movement is transmitted 546 to the base station 110.

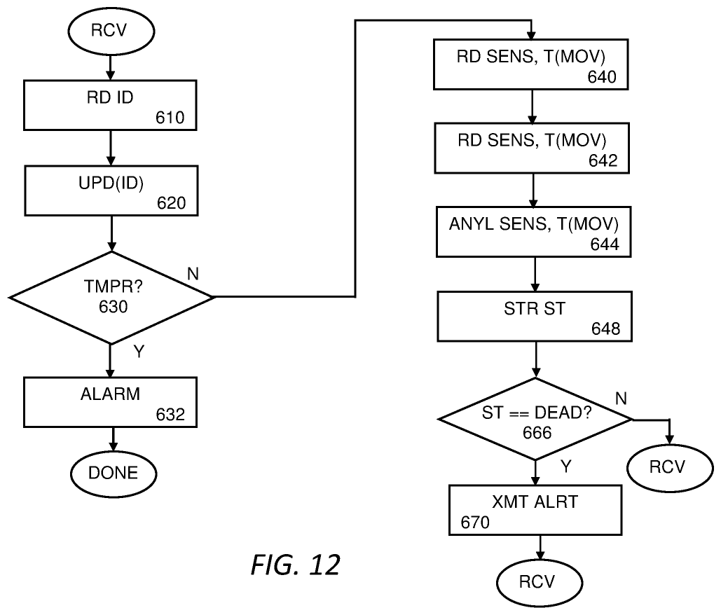

Referring to FIG. 12, an alternate flow chart of exemplary software of a base station 110 of the system for detecting life of a being is shown, working in conjunction with the flow of FIG. 11. In this example, when the base station 110 receives a transmission from any of the population of body worn-devices 40/50, the base station 110 reads 610 the identification of the body worn-devices 40/50 that sent the transmission and updates data 620 regarding that body-worn device 40/50 (e.g., updates sensor data, time since last movement, tamper status). The base station then determines if the transmission includes an indication of tampering 630 of the body worn-device 40/50 and if the transmission includes an indication of tampering 630 of the body worn-device 40/50, an alarm is issued 632, for example, an audible alarm, visual alarm, transmission of one or more messages to employees/guards (e.g., SMS messages), etc. If the transmission does not include an indication of tampering 630 of the body worn-device 40/50, then the base station reads 640 the sensor data and time since last movement from the transmission, stores 642 the sensor data and time since last movement and analyzes 644 the sensor data and time since last movement in view of previously stored sensor data to determine a status of the wearer of the body-worn device 40/50. After storing 648 the status of the wearer of the body-worn device 40/50, if the of the wearer of the body-worn device indicates 666 that the wearer is dead, in some embodiments, one or more messages are sent 670 to employees/guards (e.g., SMS messages), etc.

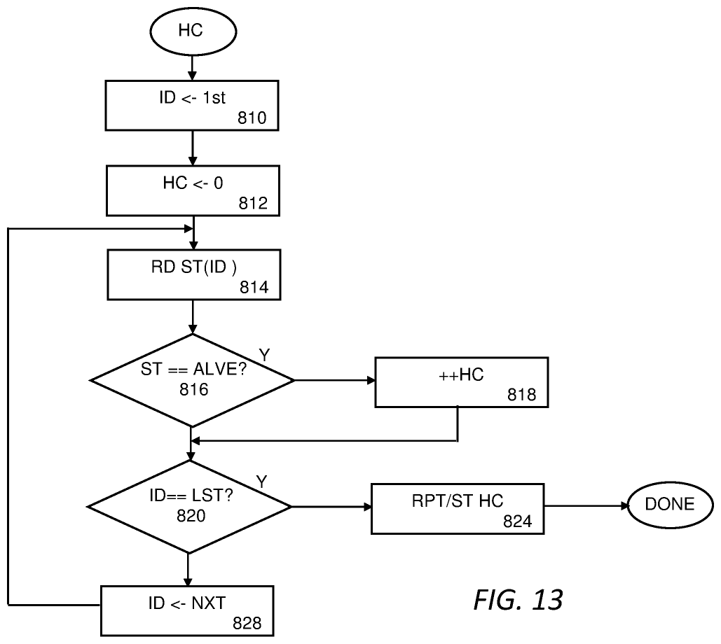

Referring to FIG. 13, a flow chart of exemplary software of a base station 110 performing a headcount of the system for detecting life of a being is shown. In this exemplary flow, a pointer (ID) is set 810 to the first person (e.g., inmate) in the population and a counter, headcount (HC), is set 812 to zero. Now, in a loop, the status of the person (e.g., inmate) currently addressed is read 814 and if the status indicates 816 that the person is alive, that person is counted 818 (e.g., the headcount is incremented by one). If the identification is not the last person 820, the next person is selected 828 and the loop continues.

It the identification is the last person 820, then the total headcount (HC) is reported 824 and any needed actions are taken if the headcount doesn't match what is expected.

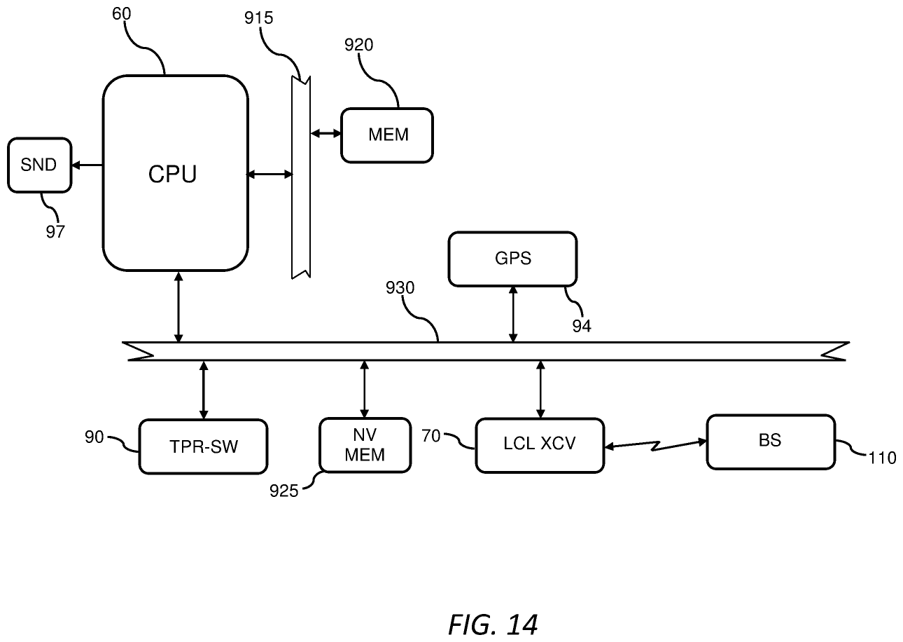

Referring to FIG. 14, a schematic view of an exemplary body worn-device 40/50 is shown. The example system represents an exemplary processor-based system housed in a body-worn device 40/50. Although, throughout this description, a processor-based system is described, it is known to implement the same or similar functionality in a system of logic or analog components providing similar functionality in an equivalent system. The source of power 98 (e.g., battery, power management, charge control, etc.) is not shown for clarity reasons.

The exemplary system of the body worn-device 40/50 is shown in its simplest form, having a single processor 60 (e.g., controller, microcontroller, microprocessor, etc.). Many different computer architectures are known that accomplish similar results in a similar fashion and the present invention is not limited in any way to any particular processing element. In exemplary circuitry of the body worn device 40, a processor 60 executes or runs stored programs that are generally stored for execution within a memory 920. The processor 60 is any processor, for example a single chip processor or the like. The memory 920 is connected to the processor by a memory bus 915 and is any memory 920 suitable for connection with the selected processor 60, such as SRAM, DRAM, SDRAM, RDRAM, DDR, DDR-2, etc. Also connected to the processor 60 is a system bus 930 for connecting to peripheral subsystems. In general, the non-volatile memory 925 is interfaced to the processor 60 through the system bus 930 and is used to store programs, executable code and data persistently. Examples of persistent storage include core memory, FRAM, flash memory, etc.

In embodiments in which Global Positioning is included, a positioning system 94 (e.g., GPS) is interfaced to the processor 60 by the system bus 930. In such, the processor controls the positioning system 94 operation by sending commands to the positioning system 94 over the system bus 930 and receiving status and data back in a similar manner (e.g., latitude and longitude).

In the tamper-resistant body-worn device 40, the tamper detection subsystem 90 is also interfaced to the processor 60 by, for example, the system bus 930 (or through an input/output port, etc.). In such, the processor controls the operation of the tamper detection subsystem 90 by sending commands to the tamper detection subsystem 90 over the system bus 930 and receiving status and data back in a similar manner (e.g., intact or “device removed from body,” etc.).

The body worn-device 40/50 communicates with the land-based system (e.g., base stations 110) through a wireless interface and wireless transceiver 70. The wireless transceiver 70 is also interfaced to the processor 60 by, for example, the system bus 930 (or through an input port, etc.). In such, the processor communicates with and controls the operation of the wireless transceiver 70 by sending commands and data to the wireless transceiver 70 over the system bus 930 and receiving status and data back in a similar manner.

Although a specific architecture is shown connecting the various subsystems 94/80/90/825/70 to the processor 60, any known interface is anticipated including, but not limited to, parallel bus architectures, serial bus architectures, parallel/serial bus architectures, input/output port interfaces, Inter-Integrated Circuit links (I²C—two-wire interface), etc.

In some embodiments, a sound emitting device 97 (not shown) is interfaced to the processor 60, in this example, through an output pin, though any form of connection is anticipated, including an interface to the system bus 930. Any type of sound emitting device 97 is anticipated such as a piezoelectric element, speaker, electromechanical vibrator, indirect sound emitter, etc. In some embodiments, the sound emitting device is driven directly by the processor 60; while in other embodiments, the sound emitting device includes driver circuitry such as an oscillator and/or power amplifier.

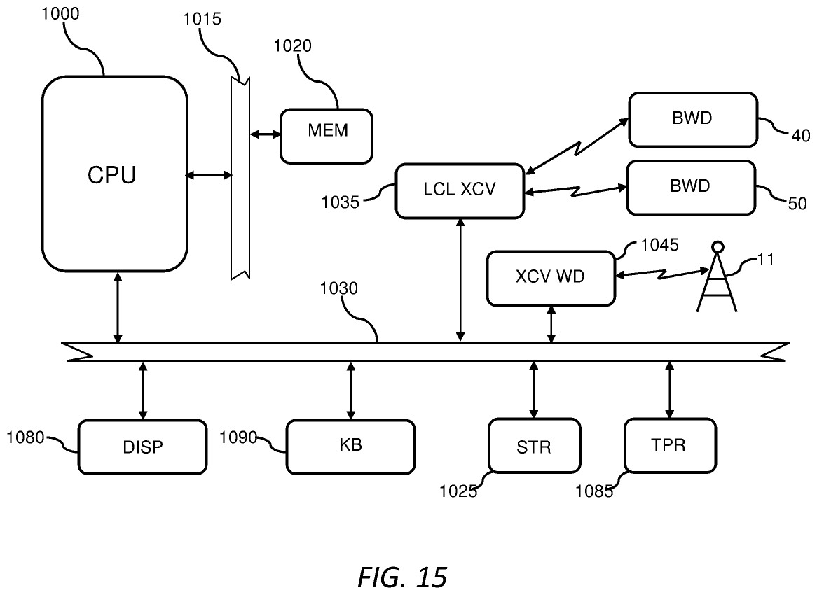

Referring to FIG. 15, a schematic view of an exemplary system of the base station 110 is shown. The example system represents an exemplary processor-based system. Although, throughout this description, a processor-based system is described, it is known to implement the same or similar functionality in a system of logic or analog components providing similar functionality in an equivalent system.

The exemplary base station 110 as shown in its simplest form has a single processor 1000, though any number are anticipated. Many different computer architectures are known that accomplish similar results in a similar fashion and the present invention is not limited in any way to any particular processing element 1000. In exemplary systems, the processor 1000 executes or runs stored programs that are generally stored for execution within a memory 1020. The processor 1000 is any suitable processor. The memory 1020 is connected to the processor 1000 by a memory bus 1015 and is any memory 1020 suitable for connection with the selected processor 1000, such as SRAM, DRAM, SDRAM, RDRAM, DDR, DDR-2, etc. Also connected to the processor 1000 is a system bus 1030 for connecting to peripheral subsystems. In general, the secondary storage 1025 is interfaced to the processor 1000 through the system bus 1030 and is used to store programs, executable code and data persistently. Examples of secondary storage 1025 include semiconductor disks, rotating media, hard disks, CD-ROM, DVD-RW, CD-RW, flash memory, etc.

The base station 110 communicates with the body-worn devices through a base station wireless transceiver 1035. The base station wireless transceiver 1035 is preferably interfaced to the processor 1000 by, for example, the system bus 1030 but alternately interfaces through an input port, a shared memory interface, etc. The processor 1000 communicates with and controls the operation of the base station wireless transceiver 1035 by sending commands and data to the base station wireless transceiver 1035 over the system bus 1030 and receiving status and data back in a similar manner.

For completeness, optional input and output devices 1080/1090 are shown such as a display 1080 and a keyboard 1090, though many different back end architectures are anticipated including one or more processors/computer systems, linked together for distributed processing and/or redundancy along with a variety of input and output devices optionally including any or all of card readers, badge readers, indicator lights, lighting control systems, audible alarms, interfaces to cell locking systems, interfaces to door locking systems, camera systems, motion detection systems, door open/closed detection systems, etc.

In some embodiments, the base station 110 includes a wide-area transceiver 1045 for communicating with the cellular system 11, for example, when sending alerts to warn of a wearer of a body-worn device 40/50 that has died or to warn of tampering. Although shown as a wireless interface, any form of wide area interfacing is anticipated, including a wired interface such as a land-line or other wired communications interface.

In some embodiments, the base station 110 also includes base station tamper detection 1085 similar or different from the tamper detection subsystem 90 of the tamper-resistant body-worn device 40. In such, intrusion into the base station 110 and/or relocation of the base station outside of a given allowed area is determined, recorded, and/or alerted. For example, in one embodiment, the base station tamper detection 1085 includes a positioning device (e.g., GPS) that constantly monitors the location of the base station 110. If the base station 110 is moved to a new location that is outside of a predetermined area, alerts are made such as transmitting an alert to other base stations 110 or repeaters, locking/encrypting data, etc. Other types of base station tamper detectors 1085 are anticipated, including, but not limited to, motion sensors, accelerometers, etc. It is also anticipated that the base station 110 be physically affixed to furniture to reduce chances of removal.

In some embodiments, the base station 110 is/are mobile devices, allowing for the base station 110 to be portable and carried by guards, staff, etc.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A system for detecting life of a being, the system comprising:
a base station, the base station includes a base station processor and a base station transceiver, the base station transceiver is operatively coupled to the base station processor;
a body-worn device, the body-worn device comprises a processor, a temperature sensor operatively coupled to the processor, a motion sensor operatively coupled to the processor, a transceiver operatively coupled to the processor, and a source of power, the source of power provides operational power to the processor, and to the transceiver, the temperature sensor configured to measure a body temperature of a wearer of the body-worn device;
the processor of the body-worn device records a time of last movement each time a signal of movement is received from the motion sensor;
the processor periodically reads the body temperature of the wearer from the temperature sensor and records the body temperature in a histogram; and
when analysis of the body temperatures recorded in the histogram indicate a change in the body temperature of greater than a predetermined rate for a predetermined time period and the time of the last movement is greater than a predetermined movement time from a current time, the processor determines that the wearer of the body-worn device is dead.

2. The system for detecting for detecting life of a being of claim 1, wherein when the processor determines that the wearer of the body-worn device is dead, the processor transmits a signal from the transceiver to the base station transceiver indicating the wearer of the body-worn device is dead.

3. The system for detecting life of a being of claim 1, wherein the body-worn device is tamper-resistant.

4. The system for detecting life of a being of claim 1, wherein the body-worn device is removable by the wearer of the body-worn device.

5. The system for detecting life of a being of claim 1, wherein the motion sensor is an accelerometer.

6. The system for detecting life of a being of claim 1, wherein the temperature sensor is a skin-contact temperature sensor.

7. The system for detecting life of a being of claim 1, wherein the temperature sensor is an infrared temperature sensor aimed at the wearer of the body-worn device.

8. The system for detecting life of a being of claim 1, wherein the predetermined movement time is configurable.

9. A system for detecting life of a being, the system comprising:
a base station, the base station including a base station processor and a base station transceiver, the base station transceiver operatively coupled to the base station processor;
a body-worn device, the body-worn device comprises a processor, a temperature sensor operatively coupled to the processor, a motion sensor operatively coupled to the processor, a transceiver operatively coupled to the processor, and a source of power, the source of power provides operational power to the processor, and to the transceiver, the temperature sensor is configured to measure a body temperature of a wearer of the body-worn device;
the processor of the body-worn device records a time of last movement each time a signal of movement is received from the motion sensor;
the processor periodically reads the body temperature of the wearer from the temperature sensor and transmits the body temperature of the wearer and the time of the last movement from the transceiver to the base station transceiver; and
when the base station processor receives the body temperature and the time of the last movement, the base station processor records the body temperature in a histogram and the base station processor analyzes the histogram to determine if the wearer of the body-worn device is dead when there was a change in the body temperature of greater than a predetermined rate for a predetermined time period and the time of the last movement is greater than a predetermined movement time from a current time.

10. The system for detecting for detecting life of a being of claim 9, wherein when the base station processor determines that the wearer of the body-worn device is dead, the base station processor transmits a signal from a wide-area transceiver to inform that the wearer of the body-worn device is dead.

11. The system for detecting life of a being of claim 9, wherein the body-worn device is tamper-resistant.

12. The system for detecting life of a being of claim 9, wherein the body-worn device is removable by the wearer of the body-worn device.

13. The system for detecting life of a being of claim 9, wherein the motion sensor is an accelerometer.

14. The system for detecting life of a being of claim 9, wherein the predetermined movement time is configurable.

15. A method of determining if a wearer of a body-worn device is dead, the body-worn device having a temperature sensor operatively coupled to the wearer and measuring a body temperature of the wearer and the body-worn device having a motion sensor, the method comprising:
(a) using the motion sensor, continuously recording a time of last movement of the body-worn device;
(b) periodically reading the body temperature from the temperature sensor; and
(c) determining a status of the wearer of the body-worn device as being dead when the body temperature of the wearer has changed by at least a predetermined amount over a predetermined period of time and the time of the last movement is greater than a predetermined time from a current time.

16. The method of claim 15, wherein the motion sensor is an accelerometer.

17. The method of claim 16, wherein the accelerometer is a three-axis accelerometer.

18. The method of claim 15, wherein when the status is dead, informing personnel that the wearer is dead.

19. The method of claim 15, wherein the body-worn device is tamper-resistant.

20. The method of claim 15, wherein the body-worn device is removable by the wearer of the body-worn device.
